(19) United States
(12) Patent Application Publication
(10) Pub. No.: US 2008/0255463 A1
(43) Pub. Date: Oct. 16, 2008

(54) Method of Obtaining an Estimation of a Person's Aortic Blood Pressure

(75) Inventors: Philip Jan Chowienczyk, London (GB); Sandrine Millasseau, Surrey (GB)

Correspondence Address: Iandioro & Teska, 260 Bear Hill Road, Waltham, MA, 02451, US

(73) Assignee: MICRO MEDICAL LTD., CHATHAM KENT (GB)

(21) Appl. No.: 11/794,275

(22) PCT Filed: Jan. 4, 2006
(86) PCT No.: PCT/GB06/00008

§ 371 Date: Feb. 22, 2008

(30) Foreign Application Priority Data
Jan. 1, 2005 (GB) 0500430.4

(51) Int. Cl. A61B 5/022; A61B 5/021; A61B 5/0215

(52) U.S. Cl. 600/486; 600/485; 600/490

(57) ABSTRACT

A method of obtaining an estimation of a person's aortic systolic blood pressure, which method comprises non-invasively measuring the person's brachial systolic blood pressure, non-invasively measuring the person's brachial diastolic blood pressure, obtaining the person's radial augmentation index by measuring the person's radial pulse waveform, and obtaining the estimation of the person's aortic systolic blood pressure from the following equation: aSBP=α+β·rA1x+γ·bSBP+δ·bDBP where the coefficients α, β, γ and δ are constants with approximate values of α=−24.2, β=0.28, γ=0.83, δ=0.17.

This invention relates to a method of obtaining an estimation of a person's aortic blood pressure.

Sustained high blood pressure is known as hypertension and it is a major cause of cardio vascular disease and death. A person's blood pressure is usually measured non-invasively. Typically, a sphygmomanometer is employed. The sphygmomanometer is externally placed around a person's arm, inflated and then the person's brachial blood pressure is read.

It is recognised that the measurement of aortic systolic blood pressure may be superior to that of brachial systolic blood pressure because blood pressure in the aorta is more closely related to the load imposed on the heart. Measurement of the aortic blood pressure rather than the brachial blood pressure may identify subjects that are at increased risk of cardiovascular disease. The measurement of the aortic systolic blood pressure may also identify person's with pseudo systolic hypertension in whom brachial systolic (maximum) blood pressure is high but aortic systolic blood pressure is normal. Assessment using brachial blood pressure alone may lead to inappropriate treatment for a person. In spite of all this, the aortic blood pressure is often not obtained because the measurement of the aortic blood pressure can usually only be made in an invasive manner using a pressure catheter in the person's aorta. Such measurements can only generally be made in a hospital and they are therefore not normally conducted due to the expense, discomfort and possible side effects imposed by such a procedure.

U.S. Pat. No. 5,265,011 and EP-A-1380254 described methods of obtaining an estimation of a person's aortic systolic blood pressure non-invasively by applying a transfer function to the pressure waveform measured from the radial artery of the person.

UK-A-0317847.2 describes a method of estimating a person's aortic systolic blood pressure by applying a transfer function to a blood pressure pulse measured at an extremity of the person using a photoplethysmograph.

The above mentioned three methods of estimating the person's aortic systolic blood pressure use a transfer function derived from data on a subset of persons. These methods assume that the transfer function is applicable to all persons. The validity of this assumption and the accuracy of the transfer function may be questioned.

It is an aim of the present invention to provide a method of obtaining an estimation of a person's aortic systolic blood pressure, which method does not require the use of a transfer function, and which method also does not require the use of a pressure catheter in the person's aorta.

Accordingly, in one non-limiting embodiment of the present invention there is provided a method of obtaining an estimation of a person's aortic systolic blood pressure, which method comprises non-invasively measuring the person's brachial systolic blood pressure, non-invasively measuring the person's brachial diastolic blood pressure, obtaining the person's radial augmentation index by measuring the person's radial pulse waveform, and obtaining the estimation of the person's aortic systolic blood pressure from the following equation:

aSBP=α+β·rA1x+γ·bSBP+δ·bDBP (1)

where the coefficients α, β, γ and δ are constants with approximate values of
α=−24.2
β=0.28
γ=0.83
δ=0.17

The radial augmentation index is the ratio of the second systolic peak to the person's radial pulse pressure (systolic pressure-diastolic pressure). The use of the equation gives an estimation of the person's aortic systolic blood pressure that is extremely accurate, for example to within 3 mm Hg.

In the above equation, the final value given for the coefficients α, β, γ and δ may vary slightly in order to provide a more accurate value of the person's aortic systolic blood pressure.

The method may be one in which the person's brachial systolic blood pressure is non-invasively measured using a sphygmomanometer. Other devices for non-invasively measuring the person's brachial systolic blood pressure may be employed.

The method may be one in which the person's brachial diastolic blood pressure is non-invasively measured using a sphygmomanometer. Other devices for non-invasively measuring the person's brachial diastolic blood pressure may be employed.

The method may be one in which the person's radial pulse waveform is measured non-invasively. In this case, the person's radial pulse waveform is preferably measured using a tonometer. Other devices for non-invasively measuring the person's radial pulse waveform may be used.

Alternatively, the person's radial pulse waveform may be measured invasively. Any invasive measurement for the person's radial pulse waveform is able to be quite simple and much less serious than the above mentioned established invasive practice of using a pressure catheter in the person's aorta. The person's radial pulse waveform is able to be measured invasively simply by placing a canula in the radial artery and attaching a pressure transducer to the canula.

An embodiment of the invention will now be described solely by way example and with reference to the accompanying drawings in which:

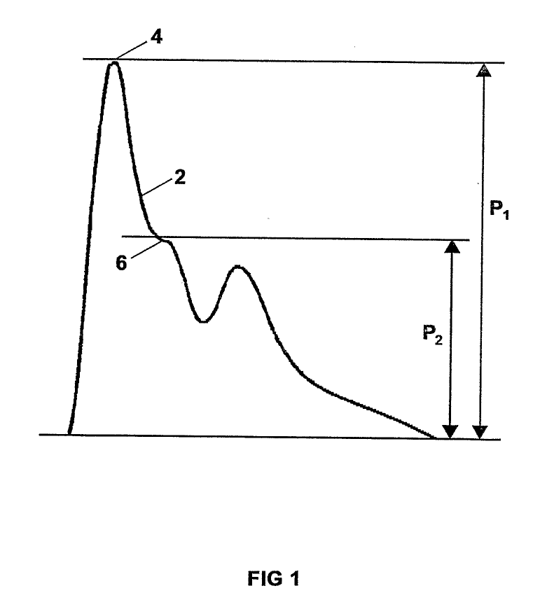

FIG. 1 is a graph of a radial pressure pulse and shows how to obtain the radial augmentation index as a ratio of the second systolic peak to the pulse pressure; and

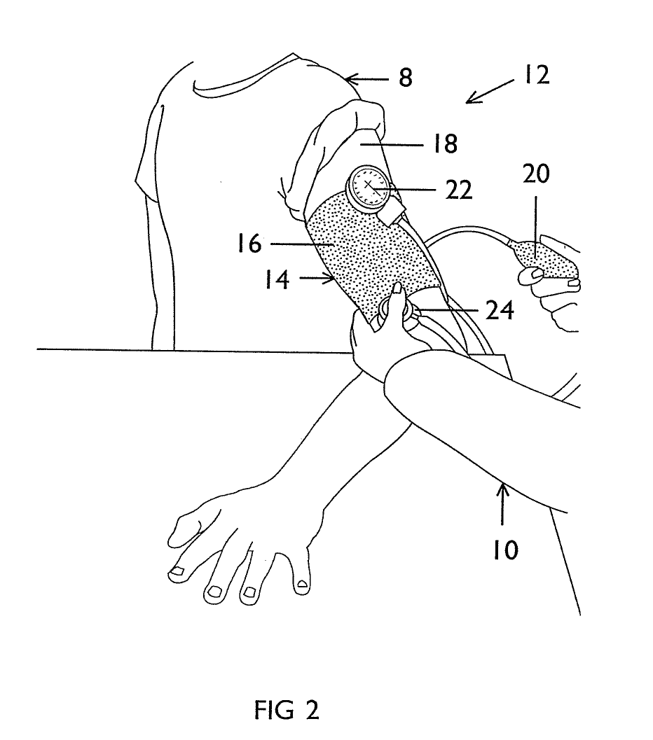

FIG. 2 shows apparatus for measuring a person's brachial systolic blood pressure and a person's brachial diastolic blood pressure.

Referring to FIG. 1, there is shown a radial pressure pulse having a first systolic peak 4, and a second systolic peak 6. The second systolic peak 6 appears somewhat as a shoulder. The first systolic peak 4 gives a pressure P₁, and the second systolic peak 6 gives a pressure P₂. The radial augmentation index is defined as the ratio of the second systolic peak 6 to the pressure pulse and may thus be defined by the following equation:

rAIx=P₂/P₁ (2)

where rAIx=radial augmentation index P₁=the first systolic peak, and P₂=the second systolic peak.

The aortic systolic blood pressure is estimated to a high degree of accuracy using the above mentioned equation No. (1).

Referring now to FIG. 2, there is shown a person having their brachial systolic blood pressure or their brachial diastolic blood pressure measured by a person 10. The person 10 is using apparatus 12 including a sphygmomanometer 14. The sphygmomanometer 14 comprises a cuff 16 which is placed around an arm 18 of the person 8. The cuff 16 is inflated using a hand pump 20. The pressure in the cuff 16 is shown on a dial 22. The apparatus 12 also comprises a stethoscope 24 which is placed over the brachial artery. By listening to the stethoscope during deflation of the cuff 16, is it possible to identify the pressure corresponding to systolic and diastolic blood pressure.

It is to be appreciated that the embodiment of the invention described above with reference to the accompanying drawings has been given by way of example only and that modifications may be affected. Thus, for example, the coefficients α, β, δ, and γ may be constants of slightly different values.

1. A method of obtaining an estimation of a person's aortic systolic blood pressure, which method comprises non-invasively measuring the person's brachial systolic blood pressure, non-invasively measuring the person's brachial diastolic blood pressure, obtaining the person's radial augmentation index by measuring the person's radial pulse waveform, and obtaining the estimation of the person's aortic systolic blood pressure from the following equation:
aSBP=α+β·rA1x+γ·bSBP+δ·bDBP
where the coefficients α, β, Y and δ are constants with approximate values of
α=−24.2=
β=0.28
Y=0.83
Δ=0.17

2. A method according to claim 1 in which the person's brachial systolic blood pressure is non-invasively measured using a sphygmomanometer.

3. A method according to claim 1 in which the person's brachial diastolic blood pressure is non-invasively measured using a sphygmomanometer.

4. A method according to claim 1 in which the person's radial pulse waveform is measured non-invasively.

5. A method according to claim 4 in which the person's radial pulse waveform is obtained using a tonometer.

6. A method according to claim 1 in which the person's radial pulse waveform is measured invasively.

7. A method according to claim 6 in which the person's radial pulse waveform is obtained using by placing a canula in the radial artery and attaching a pressure transducer to the canula.

8. (canceled)
